(19) United States
(12) Patent Application Publication
(10) Pub. No.: US 2024/0424155 A1
(43) Pub. Date: Dec. 26, 2024

(54) METHOD AND DEVICE FOR HAND SANITIZING

(71) Applicant: Resani AS, STAVANGER (NO)

(72) Inventors: Per Ivar Selvaag, STAVANGER (NO); Anne Serine Ognedal, STAVANGER (NO); Andrew St John Smith, STAVANGER (NO); Sindre Nerdal, BERGEN (NO); Ingvild Skrivervik Løken, NESØYA (NO); Øyvind Gjærevoll Schanke, STAVANGER (NO)

(73) Assignee: Resani AS, STAVANGER (NO)

(21) Appl. No.: 18/708,160

(22) PCT Filed: Nov. 9, 2022
(86) PCT No.: PCT/NO2022/050253

§ 371 Date: May 7, 2024

(30) Foreign Application Priority Data
Nov. 10, 2021 (NO) 20211353

(51) Int. Cl. A61L 2/00; A61L 2/26

(52) U.S. Cl. CPC A61L 2/0088; A61L 2/26; A61L 2202/14; A61L 2202/15

(57) ABSTRACT

A method of hand sanitation and a hand sanitation device for applying sanitation fluid to a user's hand. The method has the steps of: providing an apparatus having a volume with a cavity for placement of at least one hand, and at least one nozzle configured for spraying inside the volume; providing sanitation fluid in fluid communication with the at least one nozzle; and spraying an amount of the sanitation fluid on at least two areas on the dorsal side of a hand positioned in the volume, the at least two areas being the outside of the hand and the fingertips.

### CROSS-REFERENCE TO RELATED APPLICATIONS

This application is the U.S. national stage application of International Application No. PCT/NO2022/050253, filed Nov. 9, 2022, which international application was published on May 19, 2023, as WO 2023/085946 in the English language. The International Application claims priority to Norwegian patent application No. 20211353, filed Nov. 10, 2021. The international application and Norwegian application are both incorporated herein by reference, in their entirety.

### FIELD

The invention relates to a device for hand sanitation. More particularly, the invention relates to a device for hand sanitation where a sanitation fluid is applied to the hands in a particular pattern for compelling a hand manipulation that ensures adequate distribution of sanitation fluid on the hands following the dispensing of sanitation fluid onto the hands.

### BACKGROUND

The efficacy of hand sanitation depends on several aspects. Firstly, it depends on the sanitation efficacy of the sanitation fluid. Secondly, it depends on the amount of fluid applied on the users' hands. Lastly, a proper hand sanitation depends on the distribution of the sanitation fluid on the users' hands. The efficacy of the sanitation fluid has typically been the focus of the field. However, the application and subsequent distribution of the fluid is equally important for complete sanitation of the hands.

To ensure proper hand hygiene, it is paramount that the hands are evenly and fully covered by the sanitation fluid, be it soap or disinfectant. This is dependent on proper hand manipulation and hand rubbing after applying enough sanitation fluid to the hands. Most people follow the same hand manipulation pattern each time they are washing or disinfecting their hands, without thinking of how the rubbing is performed. Most users dispense sanitation fluid in the palm of the hand and the distribution of the fluid is therefore mostly focused on the palm of the hand, mostly leaving out other areas. Studies have shown that the areas of the hands that most often lack covering of sanitation fluid after handwashing, are the back of the hands, most notably the outside of the back of the hands, underneath the little finger, in addition to the fingertips.

Today's sanitation fluid dispensers rely on a user to dispense a fluid dose manually, which could result in too little fluid applied to the hands and therefore incomplete coverage of sanitation fluid on the hands. Automatic dispensers overcome this issue by dispensing enough sanitation fluid in each dose, but the subsequent distribution of sanitation fluid is still entirely dependent on the user. Thus, automatic dispensers do not meet the persistent and well-documented problem of incomplete distribution of hand sanitation fluid because of insufficient hand manipulation.

Different attempts have been made to overcome the problem of incomplete coverage of sanitation fluid on users' hands. These are usually based on devices aimed at spraying the sanitation fluid on the whole hand. This makes the sanitation method independent of subsequent hand rubbing to achieve a full coverage of the sanitation fluid. WO1997033527 describes a hand sanitation apparatus which covers the hands with germicidal fluid using electrostatic fluid emitters. US2007/0160515 describes a device where an antibacterial liquid is used under pressure to form a liquid mist that is circulating in two separated chambers where the hands are placed. Even though these devices solve the problem of incomplete distribution of fluid on users' hands, it may lead to an unnecessary large amount of sanitation fluid dispensed each time.

Document US 2013/0136669 A1 describes an appliance for disinfecting the hands of a user. The appliance includes a disinfection chamber, at least one opening for inserting a hand and a device for spraying a disinfectant product in the chamber to treat the entire surface of the inserted hand.

Document U.S. Pat. No. 3,918,987 A describe an apparatus comprising a pair of cavities provided in a housing for receiving hands and forearms. A plurality of nozzles within the cavities ejects streams of fluid inwardly onto the inserted hands and forearms.

Document US 2018/255984 A1 describes an apparatus for hand drying. The apparatus comprises a housing having a hand insertion opening, a pair of planar gas jets being oriented into the housing to blow off a planer aspect and a dorsal aspect of a hand inserted into the hand insertion opening.

In view of the above-described problems there is a need to further develop devices for hand sanitation available at scale.

### SUMMARY

The invention has for its object to remedy or to reduce at least one of the drawbacks of the prior art, or at least provide a useful alternative to prior art.

The object is achieved through features which are specified in the description below and in the claims that follow.

The invention is defined by the independent patent claims. The dependent claims define advantageous embodiments of the invention.

In a first aspect the invention relates to a method of hand sanitation. The method comprises the steps of:
providing an apparatus comprising a volume with a cavity for placement of at least one hand, and at least one orifice configured for applying a sanitation fluid inside the volume;
providing sanitation fluid in fluid communication with the at least one orifice; and
applying an amount of the sanitation fluid on at least two areas on the dorsal side only of a hand positioned in the volume, the at least two areas being the outside of the hand and the fingertips, the outside of the hand being the area underneath the fingers, from the middle finger to the little finger.

This method of hand sanitation may overcome the issue of uneven or lack of covering of sanitation fluid on the back of the hands and on the fingertips. By applying, such as by spraying or otherwise dispensing, sanitation fluid directly on these areas on the dorsal side of the hands, the problem areas are automatically covered, and the coverage does not depend on proper hand manipulation by the user. Furthermore, applying sanitation fluid on these dorsal areas compels the user to another hand manipulation for distribution of the fluid on the hands, which is different from the usual manipulation pattern used when the sanitation fluid is applied on the palm of the hand. When the sanitation fluid is dispensed on the outside of the dorsal side of the hand, the user is compelled to distribute the sanitation fluid from the outside of the hand towards the inside of the hand, therefore covering the whole dorsal side of the hand. In addition, this manipulation of the hands transfers sanitation fluid to the palm of the other hand. This compelled manipulation of the hands could therefore result in an efficient and even covering of the whole hand.

Preferably the method includes applying the sanitation fluid to the dorsal side of the hands only. The two areas of application may be distinct and separate, but in one embodiment the method may include applying sanitation fluid to a larger portion of the dorsal side of a user's hands, the larger portion including the two mentioned areas, i.e. the outside of the hands and the fingertips.

In order to facilitate understanding of the invention one or more expressions are further defined hereinafter.

Throughout the description and claims, the wording “outside of the hand” must be interpreted as the area on the palmar side or the dorsal side of the hand, underneath the fingers, from the middle finger to the little finger.

Throughout the description and claims, the wording “inside of the hand” must be interpreted as the area on the palmar side or the dorsal side of the hand, underneath the fingers, from the middle finger to the thumb.

Throughout the description and claims the wording “apply” as used in the context of sanitation fluid must be interpreted as dispensing the fluid onto the hands, such as by spraying through a nozzle or otherwise dispensing through an orifice.

Throughout the description and claims, the wording “orifice” must be interpreted as any opening, large or small, including a nozzle, through which sanitation fluid is dispensed onto the hands of a user.

Throughout the description and claims, the wording “cavity” must be interpreted as a hollow or open space defined by the components of the apparatus.

Throughout the description and claims, the wording “sanitation fluid” must be interpreted as a fluid appropriate for hand hygiene and hand washing. Sanitation fluids may be fluid soap, hand disinfectant or an alcohol for hand hygiene purposes. The sanitation fluid may be a spay, a mist, a gel, or a foam.

Throughout the description and claims, the wording “flat nozzle” must be interpreted as a nozzle whose spray pattern in a plane normal to the direction of application is elongated, such as substantially a line.

Throughout the description and claims, the wording “round nozzle” must be interpreted as a nozzle whose spray pattern in a plane normal to the direction of application is substantially a circle. The spray pattern may cover the whole circle, the circumference, or the circumference and the centre.

Throughout the description and claims, relative positions such as “upper” and “lower”, “front” and “back”, “horizontal” and “vertical”, etc, indicates the position that the hand sanitation device has on the subsequent figures, which is also a natural, necessary, or practical functional position.

In a second aspect the invention relates to a hand sanitation device for applying sanitation fluid to a user's hand. The hand sanitation device comprises a cavity for receiving the user's hand, wherein the cavity is formed for guiding a position of the hand. The hand sanitation device may or may not comprise a housing defining the cavity. The hand sanitation device further comprises at least one orifice for applying sanitation fluid onto the dorsal side of the hand only, on at least two areas on the dorsal side of the hand. The orifice may be connected to the housing, if present and the application may be done by spraying through a nozzle. The at least two dorsal areas are the outside of the hand and fingertips. Furthermore, the hand sanitation device comprises a means for initiating dispensing of the sanitation fluid to the user's hand. The cavity is formed for guiding the dorsal side of the hand towards the at least one orifice. It may be the shape of the cavity that guides the hand in a position by comprising slits having a tilt angle with the vertical direction of the hand sanitation device in operational use, or the cavity may comprise a guiding member protruding into the cavity for guiding a position of the hand. By guiding a position of the hand, the form of the cavity ensures that the hand is correctly positioned underneath the at least one orifice. The at least one orifice can then apply an amount of sanitation fluid onto the two areas on the dorsal side of the hand. Applying onto these areas compels the user to perform another hand manipulation that may be efficient for distributing the sanitation fluid on both sides of the hand and covering the whole hand with sanitation fluid. This manipulation may also result in fluid coverage on the areas which are often forgotten when the sanitation fluid is dispensed in the palm.

The hand sanitation device is preferably adapted to apply the sanitation fluid to the dorsal side of the hands only. The two areas of application may be distinct and separate, but in one embodiment the device may be adapted to apply sanitation fluid to a larger portion of the dorsal side of a user's hands, the larger portion including the two mentioned areas, i.e. the outside of the hands and the fingertips.

The guiding member presents a simple way of guiding the correct position of the user's hand in the cavity. A correct position of the hand inside the cavity, such as in the housing if present, may be useful for the at least one orifice to dispense on the intended area.

The guiding member may be substantially spherical for guiding the hand to a curved position around the guiding member. The spherical shape of the guiding member compels the user to curve his or her hand around the guiding member. Most people have a natural curved pose of the hands when the hands are resting. The curving of the hand around the guiding member will therefore feel natural to most people. The spherical shape may be an ellipsoid.

The hand sanitation device may comprise two orifices for applying sanitation fluid onto the at least two dorsal areas of the user's hand. One orifice may apply, such as by spraying through a nozzle, sanitation fluid on the dorsal side of the fingertips, and the other may apply, such as by spraying through a nozzle, sanitation fluid on the outside of the back of the hand. The advantage of this embodiment is that the two orifices cover one area each, leading to a proper coverage of these areas.

The at least one orifice may be movable for applying onto the at least two areas on the dorsal side of the hand. This embodiment of the invention presents an alternative way of ensuring proper coverage of sanitation fluid on the at least two dorsal areas. The orifice can “draw” a pattern from the fingertips to the outside of the hand, or from the outside of the hand towards the fingertips.

The at least one orifice may be a flat nozzle, or the at least one orifice may be a round nozzle. The flat nozzle may be advantageous for spraying the fingertips in order to cover all the fingers.

The means for initiating dispensing of the sanitation fluid to the user's hand may comprise a trigger device configured for activating application of sanitation fluid from the at least one orifice upon pressure. The advantage of this embodiment of the hand sanitation device is that the user controls when to initiate the dispensing of the sanitation fluid onto the hand.

The means for initiating dispensing of the sanitation fluid to the user's hand may comprise a sensor configured for activating application of sanitation fluid from the at least one orifice upon detection of the hand inside the housing. The advantage of a sensor for initiating application of sanitation fluid is that it does not require the user to come into contact with any surface of the hand sanitation device.

The hand sanitation device may further comprise a radio-frequency identification (RFID) reader for reading RFID tags. The RFID tags may be ported by users of the hand sanitation device, such as health care providers. The RFID reader may register each reading of a RFID tag. In an alternative embodiment, the hand sanitation device may comprise a Bluetooth® receiver while the users carry a Bluetooth® sender, such as a mobile phone or a beacon.

In another embodiment of the hand sanitation device in accordance with the invention, the hand sanitation device may comprise a tracking system for registering each use of the hand sanitation device and the time of use. The tracking system may register a time stamp of the dispensing of the sanitation fluid. The tracking system may be used together with the RFID or Bluetooth® registration to track how often persons carrying a RFID tag or Bluetooth sender is using the hand sanitation device.

The hand sanitation device may further comprise a camera configured for capturing images of the cavity. The camera may take a picture of the hand inside the cavity to identify objects on the hand that could reduce the effect of the hand sanitation, for example rings. The camera may also be used for collecting biometric data of the user. The data collected may be used to categorise and recognise the users of the hand sanitation device, for example if the user is a woman, a man, or a child.

The data from the RFID or Bluetooth® reader, from the tracking system and/or from the camera may be stored in a storage system comprised within the hand sanitation device, or in a cloud-based storage system. The collected data from the hand sanitation device may be subsequently used to gain insight of how the hand sanitation device in a particular place is used; how often, by whom, and if the users carry objects on their hand.

In an embodiment of the hand sanitation device according to the invention, the hand sanitation device may comprise a monitoring system for monitoring the condition of the hand sanitation device.

### BRIEF DESCRIPTION OF THE DRAWINGS

In the following is described examples of embodiments illustrated in the accompanying figures, wherein:

FIG. 1 shows in perspective a hand sanitation device for applying sanitation fluid to a user's hand;

FIG. 2 shows a front view of the hand sanitation device of FIG. 1;

FIG. 3 shows a side view of the hand sanitation device of FIG. 1;

FIG. 4 shows a front view of another hand sanitation device for applying sanitation fluid to a user's hand;

FIG. 5 shows a front view of yet another hand sanitation device for applying sanitation fluid to a user's hand;

FIG. 6 shows a cross section of the hand sanitation device of FIG. 5;

FIG. 7 shows a drawing of a pair of hands; and

FIG. 8 shows in perspective an alternative embodiment of a hand sanitation device for applying sanitation fluid to a user's hand; and

FIG. 9 shows in a perspective view an alternative embodiment of a cavity of a hand sanitation device.

### DETAILED DESCRIPTION OF THE DRAWINGS

Various illustrative embodiments of the present subject matter are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present subject matter will now be described with reference to the attached figures. Various systems, structures and devices are schematically depicted in the figures for purposes of explanation only and to not obscure the present disclosure with details that are well known to those skilled in the art. Nevertheless, the attached figures are included to describe and explain illustrative examples of the present disclosure. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

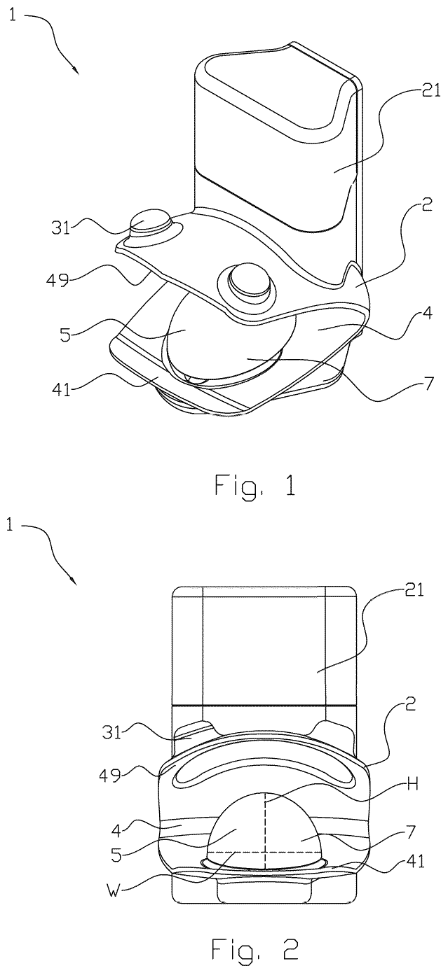

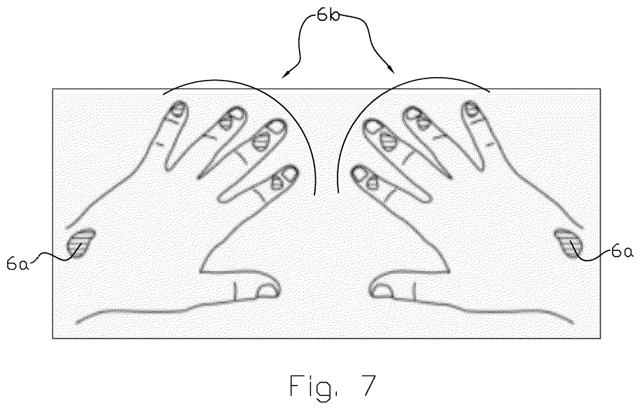

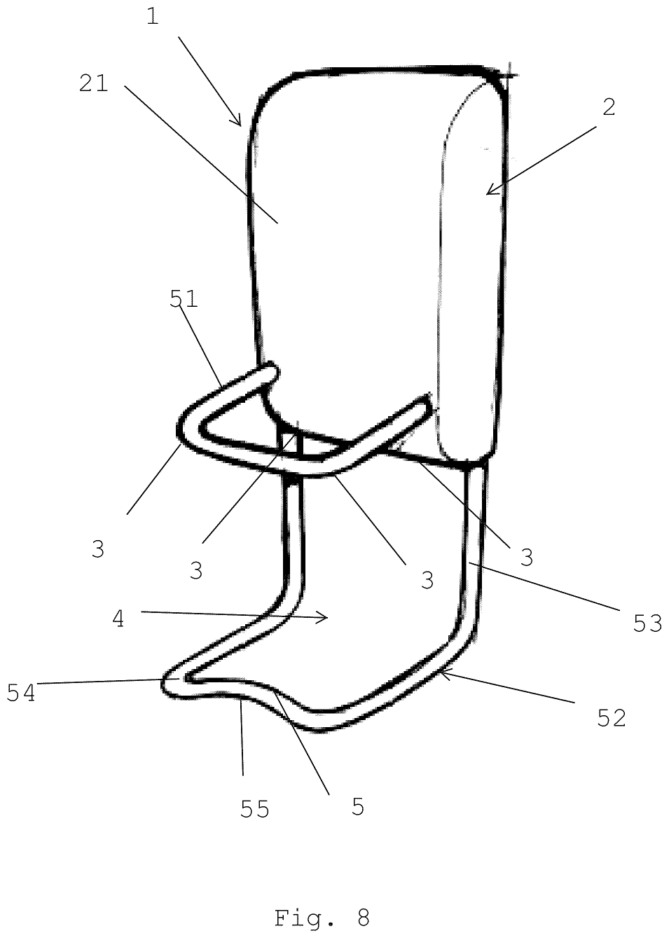

FIGS. 1 to 7 show four hand sanitation devices 1 for applying sanitation fluid to a user's hand. The hand sanitation devices 1 of FIGS. 1-6 comprise a housing 2. The housing 2 comprises a cavity 4 for receiving a user's hand. In the alternative embodiment of FIG. 7, as will be discussed below, the cavity is not defined by a rigid housing, but rather by other components of the device. The housing 2 in FIGS. 1-6 further comprises four orifices, here in the form of nozzles 3 (FIG. 5 and FIG. 6). The nozzles 3 are positioned inside nozzle housings 31, the nozzles 3 not showing in FIGS. 1 to 4. Furthermore, the housing 2 comprises a sanitation fluid reservoir 21 for containing the sanitation fluid (not shown). The nozzles 3 are in fluid communication with the sanitation fluid reservoir 21.

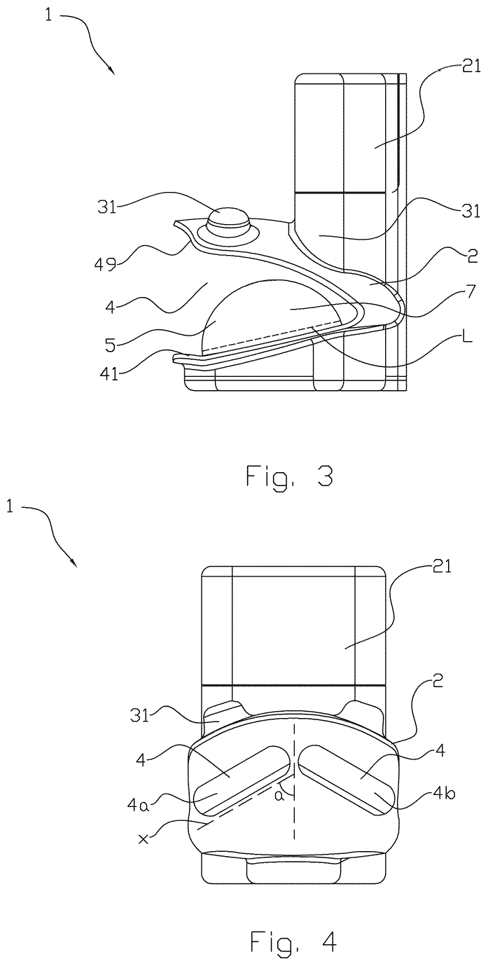

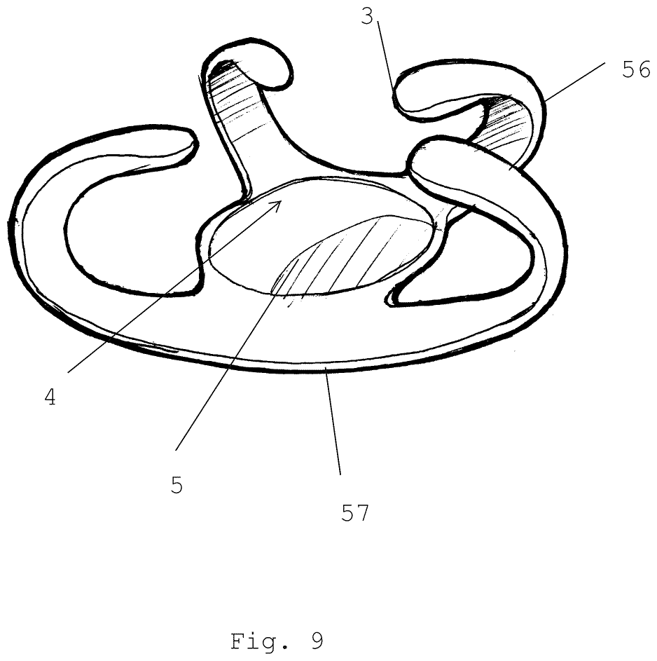

Referring to FIGS. 1 to 3 and 5 to 6, the cavity 4 comprises a guiding member 5. The guiding member 5 is positioned on the lower side 41 of the cavity 4 and has a spherical shape. This configuration of the guiding member 5 guides the user to curve his or her hands around the guiding element 5. This position of the hands places the backside of the hands underneath the nozzles 3. The guiding member 5 has an approximately ellipsoid form, shaped to mimic the form that hands usually have when resting. The width W of the guiding member 5 may be approximately 10 cm (FIG. 2). The length L of the guiding member 5 may be approximately 14 cm (FIG. 3). The height H of the guiding member 5 may be approximately 7 cm (FIG. 2). The upper side 49 of the cavity 4 is slightly curved for guiding the curved position of the hands in the cavity 4. The cavity 4 and the guiding member 5 may have other configurations and shapes, as long as the hands are guided in a correct position in respect to the nozzles 3. As also explained herein, the cavity 4 does not necessarily need to be surrounded, except from at the opening, by a rigid housing, but may instead be an open space into which the hands of a user are guided into a “correct” position for application of sanitation fluid. Orifices, such as nozzles, may be connected to rigid self-supporting conduits or an otherwise open structure arranging the nozzles/orifices similarly to what has been shown herein, but without the orifices being integrated into a part of a housing as will be explained with reference to FIGS. 8 and 9. The correct position being a position of the hands so that the spray of the nozzles 3 hit the intended area of the back of the hands.

In the embodiment shown in FIGS. 1 to 4, the two nozzles 3 in the front of the hand sanitation device 1 are positioned within a nozzle housing 31 each, while the two nozzles 3 in the back of the hand sanitation device are positioned within the housing 2. FIGS. 5 and 6 show an alternative embodiment where the two front nozzles 3 are positioned within the same nozzle housing 31, and two nozzles 3 are positioned within the housing 2.

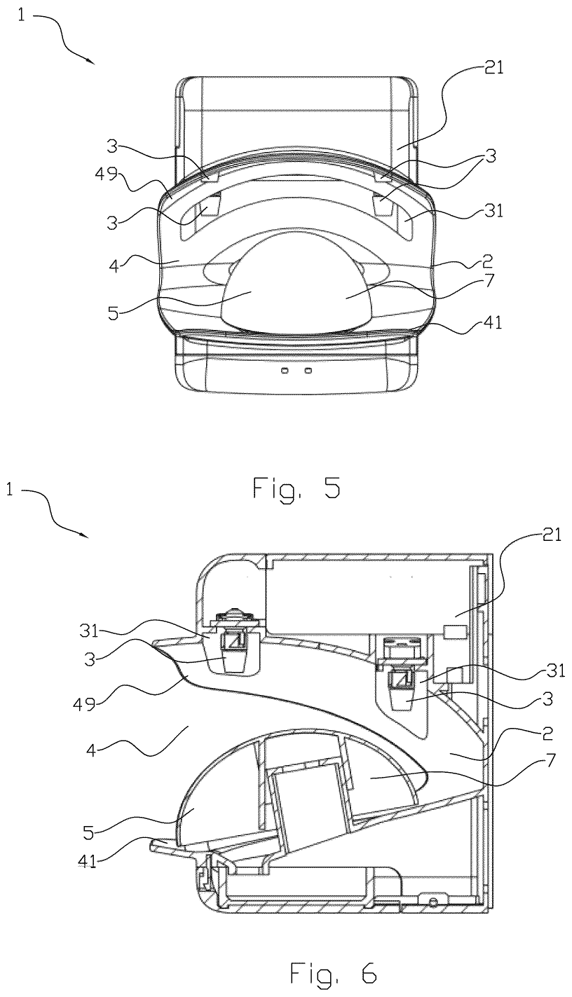

In the embodiment shown in FIGS. 1 to 3, in FIGS. 5 to 6 and in FIGS. 8 to 9, the cavity 4 is made to receive both hands of the user. The cavity 4 could in other embodiments of the invention be separated in two smaller cavities, one cavity for each hand, as shown in FIG. 4.

The cavity 4, of the hand sanitation device 1 shown in FIG. 4, comprises two slits 4a and 4b. The slit 4a is configured for receiving a user's left hand, and the slit 4b is configured for receiving the user's right hand. The slits 4a, 4b are angled in such a way that a user would place the hands in the slits 4a, 4b with the palm facing downwards. The hands are in this way placed correctly underneath the nozzles 3.

The slits 4a, 4b are elongated in a direction x. The direction x makes a tilt angle a with the vertical direction of the hand sanitation device when the hand sanitation device is in operational use. The tilt angle a can be small enough for the user to be compelled to place the hands with the palm facing downwards, so the dorsal side is facing the nozzles 3 over the hands. The tilt angle a can also be larger than 90° so that the user is compelled to place his or her hands in the slits 4a, 4b with the palm facing upwards. The slits 4a, 4b can in another embodiment be vertical. In these cases, the nozzles 3 would be placed on the outer side of the slits 4a, 4b directed inwards towards the slits 4a, 4b and towards the back of the hand of a user. When the tilt angle a is small or the tilt angle a is larger than 90°, the probability that the sanitation fluid will drain off the hands after dispensing, is higher than if the nozzles 3 are spraying downwards onto the hands.

The cavity 4 or the slits 4a, 4b should be large enough for receiving all or at least most hand sizes, and small enough to compel the right positioning of the hands in the cavity 4.

The nozzles 3 are in fluid communication with the sanitation fluid reservoir 21, for dispensing an amount of sanitation fluid on a user's hands. In the embodiments shown in FIGS. 1 to 6, the housing 2 comprises four nozzles 3, two nozzles 3 for each hand. The nozzles 3 are configured for dispensing sanitation fluid on at least two dorsal areas 6a, 6b on the user's hands (FIG. 7). The first dorsal area 6a is on the outside of the hand, and the second dorsal area 6b is on the fingertips. One nozzle 3 is spraying on the first dorsal area 6a on one hand and another nozzle 3 is spraying on the second dorsal area 6b on the hand. The dorsal areas 6a, 6b should always be covered by sanitation fluid from the nozzles 3, so the areas covered by the nozzles' 3 spray should be large enough to compensate for differences in hand sizes and hand position adjustments.

The four nozzles 3 can be flat nozzles, which could be advantageous for spraying the second dorsal area 6b. The nozzles 3 can also be round nozzles. The nozzles 3 can also be a combination of flat and round nozzles.

In another embodiment of the hand sanitation device 1, the housing 2 can comprise two orifices, such as nozzles 3, one nozzle 3 per hand. The two nozzles 3 can be movable for spraying the two dorsal areas 6a, 6b. The movable nozzles 3 can spray continuously from one dorsal area 6a, 6b to the other, or spray one of the dorsal areas 6a, 6b first, then the other dorsal area 6a, 6b. In another embodiment wherein the housing 2 comprises two nozzles 3, the two nozzles 3 can be round nozzles. The circular pattern formed by the sanitation fluid dispensed by the nozzles 3 must be large enough to cover both dorsal areas 6a, 6b. The spray pattern may cover the whole circle, the circumference, or the circumference and the centre.

The housing 2 may comprise a sensor (not shown) for detecting the presence of a hand or two hands inside the cavity 4. Upon detection, the sensor activates spraying of sanitation fluid from the nozzles 3. The sensor may detect presence of one or two hands, and after a delay, activate the spraying to allow time for the user to correctly place the hand or hands. The sensor may also be positioned in such a way that it detects if the hand or hands are in a correct position in regard to the nozzles 3, and immediately activate the spaying upon detection. The sensor may be an optical sensor.

The housing 2 may comprise a trigger device 7 configured for activating spraying of sanitation fluid from the nozzles 3 upon pressure. The trigger device 7 may for example be a mechanical button comprised within the guiding member 5, so that the user can push down the guiding member 5 to activate spraying of sanitation fluid from the nozzles 3. The trigger device 7 may also be a pedal (not shown) placed on the floor and connected to the hand sanitation device 1.

The hand sanitation device 1 may comprise a RFID or Bluetooth® reader (not shown) for reading radio-frequency identification tags or Bluetooth senders. The RFID tags or Bluetooth® senders may be carried by persons using the hand sanitation device 1, for example health care providers. The range of the RFID reader may be approximately 1 m, but could be shorter and longer than 1 m. The range of a Bluetooth® reader is typically significantly longer than that of an RFID reader. The hand sanitation device 1 may further comprise a tracking system for registering each use of the hand sanitation device and the time of use. The data from the RFID or Bluetooth® reader can be compared with the data from the tracking system for obtaining information about how often the hand sanitation device 1 is used or not used by persons who are wearing a RFID tag or Bluetooth® sender and who are within the range of the RFID or Bluetooth® reader on the hand sanitation device 1.

The hand sanitation device 1 may further comprise a camera (not shown) configured for capturing images of the cavity 4. The camera may take a picture of a user's hand inside the cavity 4 to identify objects on the hand that could reduce the effect of the hand sanitation, for example rings. The camera may also be used for collecting biometric data of the user.

The hand sanitation device 1 may comprise a monitoring system (not shown) for monitoring the condition of the hand sanitation device 1. The monitoring system may monitor the level of sanitation fluid inside the fluid reservoir 21. If the level of sanitation fluid is lower than a pre-set threshold, the monitoring system may notify operators by for example an indicator light (not shown) for providing feedback to an operator or user of the hand sanitation device 1.

FIG. 8 shows an alternative embodiment of a sanitation device 1 according to the invention, in which the device 1 is mounted to a wall 50. Two, not shown orifices, here in the form of nozzles 3, are integrated into a distal portion of a horizontal first rail 51, one substantially at each bend, while two additional nozzles 3 are integrated at the lower portion of the housing 2, substantially vertically below the proximal end of the horizontal first rail 51 at the housing 2. The cavity 4 is defined by the open space below the first rail 51 and a second rail 52 extending vertically downwardly in a vertical section 53 from the lower end of the housing 2, along the wall 50. The second rail 52 is bent into a substantially horizontal, lower portion 54, extending horizontally outwardly from the wall 50, the lower portion 54 of the second rail 52 defining the bottom portion of the cavity 4. The horizontal, lower portion 54 is shaped with an upwardly curved central portion 55 functioning as a guiding member 5.

An alternative arrangement of a cavity 4 as defined by means of four nozzles 3 in a “tentacle-like” arrangement is shown in FIG. 9. The tentacles 56 curve upwardly from a base plate 57 with the nozzles 3 pointing obliquely, downwardly towards the base plate 57. The base plate 57 is shaped with a guiding member 5 in the form of a hemisphere, for more clearly guiding the hands of a user to a curved position around the guiding member 5. In embodiments shown in FIG. 9, conduits connecting the nozzles 3 with the (not shown) liquid reservoir 21 are extending inside the tentacles 56. In an alternative, not shown embodiment, the conduits may be self-supporting and extending openly from the liquid reservoir 21 to the nozzles 3 without being surrounded by additional, external structure.

The person skilled in the art may easily find alternative solutions for the hand sanitation device. The invention covers all these variants as long as they are covered by the independent claims. No limitations are intended to the details of construction or design herein shown, other than as described in the claims below. It is therefore evident that the particular embodiments disclosed above may be altered or modified and all such variations are considered within the scope of the invention. Accordingly, the protection sought herein is as set forth in the claims below.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The mere fact that certain measures are recited in mutually different dependent claims does not indicate that a combination of these measures cannot be used to advantage. The invention may be implemented by means of hardware comprising several distinct elements, and by means of a suitably programmed computer. In the device claims enumerating several means, several of these means may be embodied by one and the same item of hardware.

1. A method of hand sanitation, the method comprising the steps of:
providing an apparatus comprising a volume with a cavity for placement of at least one hand, and at least one orifice configured for applying a sanitation fluid inside the volume;
providing sanitation fluid in fluid communication with the at least one orifice; and
applying an amount of the sanitation fluid on at least two areas on the dorsal side only of a hand positioned in the volume, the at least two areas being the outside of the hand and the fingertips, the outside of the hand being the area underneath the fingers, from the middle finger to the little finger.

2. A hand sanitation device for applying sanitation fluid to a user's hand, the hand sanitation device comprising:
a cavity for receiving the user's hand;
at least one orifice for applying sanitation fluid onto the dorsal side of the hand only on at least two dorsal areas of the hand, the at least two areas being the outside of the hand and the fingertips, the outside of the hand being the area underneath the fingers, from the middle finger to the little finger; and
a means for initiating dispensing of the sanitation fluid onto the user's hand;
wherein the cavity is formed for guiding the dorsal side of the hand towards the at least one orifice by:
comprising slits having a tilt angle with the vertical direction of the hand sanitation device in operational use, for guiding a position of the hand; or
comprising a guiding member for guiding a position of the hand.

3. (canceled)

4. The hand sanitation device according to claim 2, wherein the guiding member is substantially spherical for guiding the hand to a curved position around the guiding member.

5. The hand sanitation device according to claim 2, comprising two orifices for applying sanitation fluid onto the at least two dorsal areas of the hand.

6. The hand sanitation device according to claim 2, wherein the at least one orifice is movable for applying the sanitation fluid to the at least two dorsal areas of the hand.

7. The hand sanitation device according to claim 2, wherein the at least one orifice is a flat nozzle.

8. The hand sanitation device according to claim 2, wherein the at least one orifice is a round nozzle.

9. The hand sanitation device according to claim 2, wherein the means for initiating dispensing of the sanitation fluid comprises a trigger device configured for activating application of sanitation fluid from the at least one orifice upon pressure.

10. The hand sanitation device according to claim 2, wherein the means for initiating dispensing of the sanitation fluid comprises a sensor configured for activating application of sanitation fluid from the at least one orifice upon detection of the hand.

11. The hand sanitation device according to claim 2, further comprising a radio-frequency identification reader or a Bluetooth reader for reading radio-frequency identification tags or a Bluetooth sender, respectively.

12. The hand sanitation device according to claim 2, further comprising a tracking system for registering each use of the hand sanitation device and the time of use.

13. The hand sanitation device according to claim 2, comprising a housing with a camera configured for capturing images of the cavity.

14. The hand sanitation device according to claim 2, further comprising a monitoring system for monitoring the condition of the hand sanitation device.

15. The hand sanitation device according to claim 4, comprising two orifices for applying sanitation fluid onto the at least two dorsal areas of the hand.

16. The hand sanitation device according to claim 4, wherein the at least one orifice is movable for applying the sanitation fluid to the at least two dorsal areas of the hand.

17. The hand sanitation device according to claim 5, wherein the at least one orifice is movable for applying the sanitation fluid to the at least two dorsal areas of the hand.

18. The hand sanitation device according to claim 4, wherein the at least one orifice is a flat nozzle.

19. The hand sanitation device according to claim 5, wherein the at least one orifice is a flat nozzle.
